(19) United States
(12) Patent Application Publication
(10) Pub. No.: US 2023/0414208 A1
(43) Pub. Date: Dec. 28, 2023

(54) SYSTEMS AND METHODS FOR DISSECTING CATHETERS

(71) Applicant: COVIDIEN LP, Boulder, CO (US)

(72) Inventor: Scott Frushour, Boulder, CO (US)

(73) Assignee: COVIDIEN LP, Boulder, CO (US)

(21) Appl. No.: 17/972,207

(22) Filed: Oct. 24, 2022

Related U.S. Application Data
(60) Provisional application No. 63/354,253, filed on Jun. 22, 2022.

(51) Int. Cl. A61B 10/06; A61B 18/12; A61B 18/14

(52) U.S. Cl. CPC A61B 10/06; A61B 18/12; A61B 18/1445; A61B 2018/00595

(57) ABSTRACT

Systems, instruments, and methods for operating a catheter. The methods comprise: sliding a scissor actuator in a first direction within the catheter's tube; causing pushing forces to be applied to scissor forceps as the scissor actuator slides in the first direction; using the pushing forces to transition the scissor forceps from a first position to a second position (the scissor forceps comprising a pair of pivotally coupled engagement members at least partially disposed within the tube at a distal end of the catheter, each engagement member comprising a free distal end and a proximal end coupled to the scissor actuator); sliding the scissor actuator in a second opposing direction within the catheter's tube; causing pulling forces to be applied to the scissor forceps as the scissor actuator slides in a second opposing direction; and using the pulling forces to transition scissor forceps back to the first position.

### CROSS-REFERENCE TO RELATED APPLICATIONS

The present application claims priority to U.S. Provisional Application Ser. No. 63/354,253 which was filed on Jun. 22, 2022 and entitled “Systems and Methods for Dissecting Catheters”. The content of this Provisional Application is incorporated herein in its entirety.

### BACKGROUND

Catheter intervention in medical procedures continues to grow. Newer catheters are robotically controlled and include cameras to enable direct visualization of tissue that is being manipulated. Tools are typically designed to be atraumatic (i.e., cause minimal tissue injury) unless directly engaged with the tissue. Common tools include brushes, needles, electrocautery devices, and forceps.

Forceps are a handheld hinged instrument used for grasping and holding objects during surgery or laboratory experiments. Surgical forceps are often made of high-grade metal such that they can withstand repeated sterilization in autoclaves. The metal can include, but is not limited to, carbon steel, stainless steel, titanium, chromium and vanadium alloys. These surgical forceps are non-disposable forceps. The surgical forceps may be non-locking with a hinge at a distal end opposed from the proximal grasping end. Some forceps are blunt until opened while others may be more pointed to enable dissection capabilities. When opened, the distal end is separated into two spaced apart engagement members. Each grasping member can have fenestrated teeth or clamping effects.

### SUMMARY

The present disclosure relates to implementing systems and methods for operating a dissecting catheter. The methods comprise: sliding a scissor actuator in a first direction within a tube of the catheter; causing pushing forces to be applied to scissor forceps as the scissor actuator slides in the first direction; using the pushing forces to transition the scissor forceps from a first position (e.g., a closed position) to a second position (e.g., an open position); sliding the scissor actuator in a second opposing direction within the tube of the catheter; causing pulling forces to be applied to the scissor forceps as the scissor actuator slides in a second opposing direction; and using the pulling forces to transition scissor forceps back to the first position.

The scissor forceps comprise a pair of pivotally coupled engagement members at least partially disposed within the tube at a distal end of the catheter. Each engagement member comprises a free distal end and a proximal end coupled to the scissor actuator.

In some scenarios, the methods also comprises: allowing the proximal ends of the engagement members to travel outside of the tube when the scissor forceps is being transitioned to the second position; grasping or cutting tissue using teeth of the engagement members; holding a tissue sample in an interior cavity of the engagement member(s); sliding sensor(s), fluid delivery device(s) and fluid removal device(s) through an interior cavity of the scissor actuator(s) and the scissor forceps; generating sensor data by a sensor at least partially disposed in the engagement member(s) of the scissor forceps; applying heat or electrical current to tissue using electrode(s) coupled to the engagement member(s) of the scissor forceps; using a blade provided in an internal cavity of the engagement member(s) to cut tissue; using the scissor actuator to supply an electric current or power to electronic component(s) of the scissor actuator; and/or using plates respectively disposed in the engagement members to deliver electrical current to tissue.

The present document also concerns a catheter. The catheter comprises a tube, a scissor actuator slidingly disposed in the tube, and scissor forceps. The scissor forceps comprise a pair of pivotally coupled engagement members at least partially disposed within the tube at a distal end of the catheter. Each engagement member comprises a free distal end and a proximal end coupled to the scissor actuator.

Pushing forces are applied to the scissor forceps when the scissor actuator slides within the tube in a first direction. Pulling forces are applied to the scissor forceps when the scissor actuator slides within the tube in a second opposing direction. The pushing forces cause the scissor forceps to transition from a first position (e.g., a closed position) to a second position (e.g., an open position). The pulling forces cause the scissor forceps to transition back to the first position.

Openings may be formed in the tube through which the proximal ends of the engagement members travel out of the tube when the scissor forceps is being transitioned to the second position. The engagement members may comprise teeth for grasping or cutting tissue and/or an interior cavity in which a tissue sample may be held.

Sensor(s), fluid delivery device(s) and/or fluid removal device(s) may be slidingly disposed in an interior cavity of the scissor actuator and/or the scissor forceps. Additionally or alternatively, sensor(s) is(are) at least partially disposed in the engagement member(s) of the scissor forceps. Electrode(s) may be coupled to the engagement member(s) of the scissor forceps. The electrode(s) may be configured to apply heat or electrical current to tissue. A blade may be provided in an internal cavity of the engagement member(s). Plates may be respectively disposed in the engagement members and configured to deliver electrical current to tissue. The scissor actuator may have a dual purpose of (i) actuating the scissor forceps and (ii) supplying an electric current or power to at least one electronic component of the scissor actuator.

The foregoing and other objects, features and advantages of the invention will be apparent from the following more particular descriptions of exemplary embodiments of the invention as illustrated in the accompanying drawings wherein like reference numbers generally represent like parts of the disclosure.

### BRIEF DESCRIPTION OF DRAWINGS

The following drawings are illustrative of particular embodiments of the present disclosure and therefore do not limit the scope of the present disclosure. The drawings are not to scale and are intended for use in conjunction with the explanations in the following detailed description.

FIG. 1 provides an illustration of a catheter with scissor forceps at a distal end thereof.

FIG. 2 provides a cross-sectional view of the catheter of FIG. 1 with the scissor forceps in a closed position.

FIG. 3 provides a cross-sectional view of the catheter of FIG. 1 with the scissor forceps in an open position.

FIG. 4 provides illustrations showing another catheter design with scissor forceps at a distal end thereof.

FIG. 5 provides illustrations showing another catheter design with scissor forceps at a distal end thereof and at least one of sensor(s) and fluid delivery device(s).

FIG. 6 provides an illustration showing another catheter design with scissor forceps at a distal end thereof and at least one of sensor(s) and electrode(s).

FIG. 7 provides an illustration showing another catheter design with a blade provided with the scissor forceps at a distal end thereof.

FIGS. 8-11 each provides illustrations showing different architectures for engagement members of scissor forceps.

FIG. 12 provides an illustration showing another architecture for an engagement member of scissor forceps.

FIG. 13 provides an illustration of a system.

FIG. 14 provides an illustration of a computing device.

FIG. 15 provides a flow diagram of an illustrative method for operating a catheter.

FIGS. 16-17 each provide an illustration of another architecture for a catheter with scissor forceps.

### DETAILED DESCRIPTION

The following discussion omits or only briefly describes certain conventional features related to surgical systems for treating the spine, which are apparent to those skilled in the art. It is noted that various embodiments are described in detail with reference to the drawings, in which like reference numerals represent like parts and assemblies throughout the several views. Reference to various embodiments does not limit the scope of the claims appended hereto. Additionally, any examples set forth in this specification are intended to be non-limiting and merely set forth some of the many possible embodiments for the appended claims. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

In the airways of individuals, more than half of all lesions lie outside the airway walls and provide no visual evidence of their presence. It may be advantageous to provide a forceps design that allows for traumatic dissection of an airway wall to allow a physician direct visualization of the tumor or nodule to be engaged for diagnostic and/or therapeutic purposes.

Embodiments of the present disclosure generally relate to implementing systems and methods for operating a catheter. The methods comprise: sliding a scissor actuator in a first direction within a tube of the catheter; causing pushing forces to be applied to scissor forceps as the scissor actuator slides in the first direction; using the pushing forces to transition the scissor forceps from a first position (e.g., a closed position) to a second position (e.g., an open position); sliding the scissor actuator in a second opposing direction within the tube of the catheter; causing pulling forces to be applied to the scissor forceps as the scissor actuator slides in a second opposing direction; and using the pulling forces to transition scissor forceps back to the first position.

The scissor forceps comprise a pair of pivotally coupled engagement members at least partially disposed within the tube at a distal end of the catheter. Each engagement member comprises a free distal end and a proximal end coupled to the scissor actuator.

In some scenarios, the methods also comprises: allowing the proximal ends of the engagement members to travel outside of the tube when the scissor forceps is being transitioned to the closed position; grasping or cutting tissue using teeth of the engagement members; holding a tissue sample in an interior cavity of the engagement member(s); sliding sensor(s), fluid delivery device(s) and fluid removal device(s) through an interior cavity of the scissor actuator(s) and the scissor forceps; generating sensor data by a sensor at least partially disposed in the engagement member(s) of the scissor forceps; applying heat or electrical current to tissue using electrode(s) coupled to the engagement member(s) of the scissor forceps; using a blade provided in an internal cavity of the engagement member(s) to cut tissue; using the scissor actuator to supply an electric current or power to electronic component(s) of the scissor actuator; and/or using plates respectively disposed in the engagement members to deliver electrical current to tissue.

The present document also concerns a catheter. The catheter comprises a tube, a scissor actuator, and scissor forceps. The scissor actuator is slidingly disposed in the tube. The scissor forceps comprise a pair of pivotally coupled engagement members at least partially disposed within the tube at a distal end of the catheter. Each engagement member comprises a free distal end and a proximal end coupled to the scissor actuator.

Pushing forces are applied to the scissor forceps when the scissor actuator slides within the tube in a first direction. Pulling forces are applied to the scissor forceps when the scissor actuator slides within the tube in a second opposing direction. The pushing forces cause the scissor forceps to transition from a first position (e.g., closed position) to a second position (e.g., an open position). The pulling forces cause the scissor forceps to transition back to the first position.

Openings may be formed in the tube through which the proximal ends of the engagement members travel out of the tube when the scissor forceps is being transitioned to the second position. The engagement members may comprise teeth for grasping or cutting tissue and/or an interior cavity in which a tissue sample may be held.

Sensor(s), fluid delivery device(s) and/or fluid removal device(s) may be slidingly disposed in an interior cavity of the scissor actuator and/or the scissor forceps. Additionally or alternatively, sensor(s) is(are) at least partially disposed in the engagement member(s) of the scissor forceps and/or electrode(s) is(are) coupled to the engagement member(s) of the scissor forceps. The electrode(s) may be configured to apply heat or electrical current to tissue and/or apply a radiating electric and/or electromagnetic field to induce cellular change to the tissue (for example, Pulsed Field Ablation and/or Microwave Ablation). A blade may be provided in an internal cavity of the engagement member(s), and/or plates may be respectively disposed in the engagement members and configured to deliver electrical current to tissue. The scissor actuator may have a dual purpose of (i) actuating the scissor forceps and (ii) supplying an electric current or power to at least one electronic component of the scissor actuator.

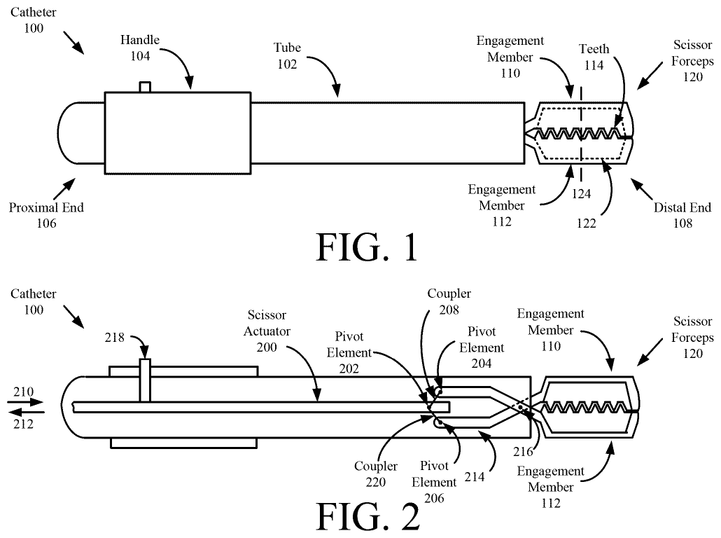

Referring now to FIG. 1, there is provided an illustration of a catheter 100. Catheter 100 comprises a tube 102 for being at least partially inserted through a narrow opening into a body cavity during a medical procedure. For example, the catheter 100 may be used to remove fluid from a patient's bladder. The present solution is not limited to the particulars of this example.

The tube 102 can be flexible or semi-rigid. The tube 102 may be disposable or non-disposable. The tube 102 may be formed of, for example, a rubber, a plastic, a polymer, an impregnated polymer, a cloth, a braided material and/or a metal. The metal can include, but is not limited to, stainless steel, titanium and/or other alloy which is resistant to corrosion.

A handle 104 may optionally be provided with the catheter 100. The handle 104 resides at a proximal end 106 of the tube 102. The tube 102 and handle 104 may be integrally formed as one part (not shown), or alternatively may be coupled to each other via a coupling means as shown in FIGS. 1-2. The coupling means can include, but is not limited to, weld(s), adhesive(s), chemical bond(s) and/or mechanical coupler(s). In some scenarios, the handle is removably coupled to the tube 102 such that it can be reused even when the tube is disposable. Accordingly, the mechanical coupler may comprise clamp(s), latch(es), and/or screw(s). The handle 104 can be formed of any material suitable for a given application. The material can include, but is not limited to, rubber, plastic, polymer, and/or metal (for example, stainless steel, titanium and/or other alloy which is resistant to corrosion). The handle 104 has a size and shape that allows an individual to easily grip and manipulate the catheter 100 without discomfort.

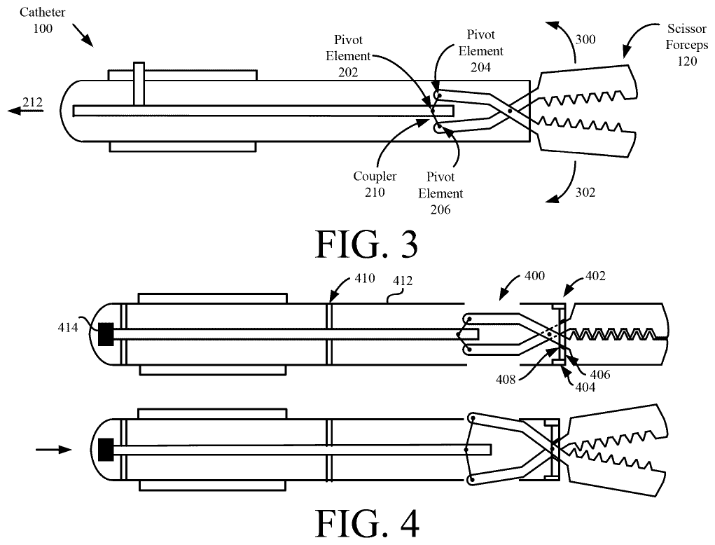

Scissor forceps 120 are provided at a distal end 108 of the catheter 100. The scissor forceps 120 may be formed of a rigid material such as plastic and metal. The metal can include, but is not limited to, stainless steel, titanium and/or other alloy which is resistant to corrosion. The scissor forceps 120 are generally configured to grip and/or cut tissue of an individual. In this regard, the scissor forceps 120 comprise engagement members 110, 112 which are pivotally coupled to each other via pivot element 216 so that they are transitionable between a closed positions shown in FIGS. 1-2 and an open position shown in FIG. 3. Center axis 124 of the engagement members 110, 112 are aligned with each other when the scissor forceps 120 are in the closed position as shown in FIG. 1.

In the closed position, teeth 114 of the engagement members 110, 112 abut and are in contact with each other. The present solution is not limited in this regard. For example, in other scenarios, the teeth may be adjacent to one another but not necessarily contact each other. Alternatively, the engagement members may be absent of any teeth. In the teeth scenario, spacers may be provided to ensure that the opposing sets of teeth are spaced a certain distance from each other when the engagement members 110, 112 are in the closed position. The teeth 114 may be dull or sharp depending on a given application for the catheter 100. Each engagement member 110, 112 may be hollow such that an interior cavity 122 is provided for holding and removing samples (for example, tissue samples) from the individual.

The scissor forceps 120 are actuated by a scissor actuator 200. Scissor actuator 200 can include, but is not limited to, a post, a bar, a wire, a cable, a hollow tube and/or pin/slot configuration (for example, a pin at 202 inserted into a slot on 214). The scissor actuator 200 is movable in two opposing directions 210 and 212, and pivotally coupled to proximal ends 214 of the engagement members 110, 112 via pivot element 202, 204, 206 and couplers 208, 220. Movement of the scissor actuator 200 in directions 210, 212 can be facilitated by a trigger mechanism 218 provided with the handle 104 or other means (for example, via the supply of electrical current to a shape memory material 414 coupled to the scissor actuator 200). Couplers 208, 220 can include, but are not limited to, wires, posts and/or plates. The couplers 208, 220 apply a pushing force respectively to the proximal ends 214 of the engagement members 110, 112 so as to cause them to move away from each other when the scissor actuator 200 slides in direction 210 as shown in FIG. 3. In contrast, the couplers 208, 220 apply a pulling force respectively to the proximal ends 214 of the engagement members 110, 112 so as to cause them to move towards each other when the scissor actuator 200 slides in direction 212. The pushing and pulling forces facilitate transitioning of the engagement members 110, 112 between their open and closed positions.

In some scenarios, the tube 102 is designed so that the proximal ends 214 of the engagement members 110, 112 remain therein throughout use of the catheter 100. In other scenarios shown in FIG. 4, the tube comprises slits or other apertures 400 formed therein through which the proximal ends 214 of the engagement members 110, 112 can travel when the scissor forceps 120 are being actuated.

The scissor forceps 120 are coupled to the tube 102 via any suitable means selected in accordance with a given application of the catheter 100. In some scenarios, the scissor forceps 120 are securely coupled to the tube 102 by pin(s) (which may or may not comprise pivot element 216). In other scenarios, the scissor forceps 120 are removably coupled to the tube 102 such that they can be reused even when the tube 102 is disposable. This coupling of the scissor forceps 120 to the tube 102 can be achieved via pin(s) (which may or may not comprise pivot element 216), a cap and/or stand-off structures.

An illustrative cap 402 is shown in FIG. 4. The cap 402 comprises a coupling rim 404 and a flexible interior member 406. The coupling rim 404 may frictionally engage a sidewall 412 of the tube. An aperture 408 is formed in the center of the flexible interior member 406 through which the proximal ends 214 of the engagement members 110, 112 can pass. The aperture 408 enlarges when the engagement members 110, 112 transition to their open position, and become smaller when the engagement members 110, 112 transition to their closed position. The present solution is not limited to this cap architecture shown in FIG. 4.

Illustrative stand-off structures 410 are also shown in FIG. 4. Each stand-off structure 410 extends from a sidewall 412 of the tube towards a center of the tube. Each stand-off may be a post or a disc with aperture(s) formed therein. Any number of stand-off structures 410 can be provided in accordance with a given application. In the case where multiple stand-off structures 410 are provided, they may be equally or unequally spaced along a length of the tube. The stand-off structures 410 are designed to allow the scissor actuator to slide thereon or therethrough without movement towards the sidewall 412 of the tube, i.e., such that the position of the scissor actuator remains the same relative to the tube throughout use of the catheter and/or scissor forceps.

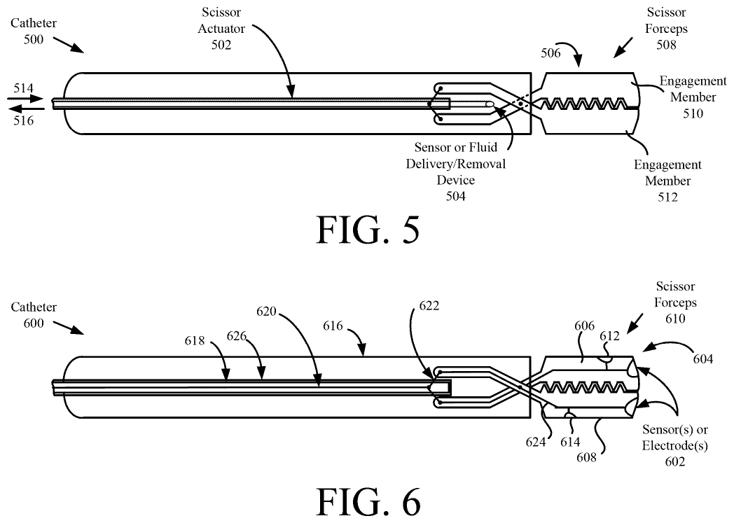

FIG. 5 shows a catheter 500 which is configured to generate sensor data, take tissue samples and/or deliver fluid during a medical procedure. In this regard, the catheter 500 may be provided with a hollow scissor actuator 502 through which a sensor or fluid delivery/removal device 504 can pass. The sensor or fluid delivery/removal device 504 is able to slidingly move in two opposing directions 514, 516 within the hollow scissor actuator 502 such that it can be selectively extended adjacent to, within and/or out of the distal ends 506 of the engagement members 510, 512 of the scissor forceps 508. The sensor device can include, but is not limited to, temperature sensor(s), camera(s), shape sensor(s), liquid sensor(s), odor sensor(s), navigation sensor(s) and/or density sensor(s). The fluid delivery device can include a tube coupled to a pump (not shown), a needle port (not shown) and/or valve (not shown). The fluid removal device can include, but is not limited to, a tube coupled to a vacuum (not shown), a needle port (not shown) and/or valve (not shown).

FIG. 6 shows a catheter 600 which is configured to generate sensor data and/or electrical current through tissue during a medical procedure. In this regard, the sensor(s) and/or electrode(s) 602 may be disposed at the distal tip(s) 604 of the engagement member(s) 606, 608 of the scissor forceps 610 and/or disposed at other locations 612, 614 along elongate sidewalls of the engagement member(s) 606, 608 of the scissor forceps 610. The sensor(s) and/or electrode(s) are electrically connected to external device(s) (for example, a power source and/or processor) via conductive cable(s), etched trace(s) and/or wire(s). The conductive cable(s) and/or wire(s) can extend through the tube 616, scissor forceps 610 and/or scissor actuator 618. The etched trace(s) can, for example, run an interior length of the tube 616 or the body 618.

In some scenarios, wire(s) 620, 624 is(are) disposed in or integrated with the scissor actuator 618, couplers 622 and scissor forceps 610 as shown in FIG. 6. These components 618, 622, 610 can be formed of, encompassed or otherwise covered with a non-conductive and/or insulative material 626 (for example, rubber). Alternatively, the scissor actuator 618, couplers 622 and/or scissor forceps 610 act as the electrically conductive members for supplying power, voltage, current and/or other signals to the sensor(s) and/or electrode(s) 602. Consequently, these components 618, 622, 610 can have dual purposes.

For example, the scissor actuator 618 can have the dual purposes of (1) actuating the scissor forceps 610 and/or (2) facilitating the supply of power and/or signals to/from the sensor(s)/electrode(s) 602. The coupler(s) 622 have the dual purposes of (1) facilitating actuation of the scissor forceps 610 via movement of the scissor actuator 618 and/or (2) facilitating the supply of power and/or signals to/from the sensor(s)/electrode(s) 602. The scissor forceps have the dual purposes of (1) facilitating the holding or cutting of tissue and (2) facilitating the supply of power and/or signals to/from the sensor(s)/electrode(s) 602. The present solution is not limited to the particulars of this example.

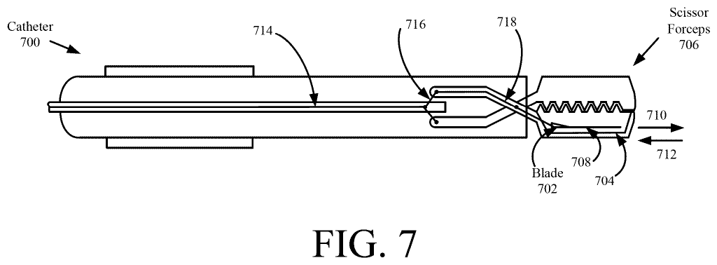

FIG. 7 provides an illustration of another catheter 700 with one or more blade(s) 702. The blade(s) 702 is(are) disposed in an interior cavity 704 of the scissor forceps 706. The blade(s) 702 may be configured to entirely reside in the interior cavity 704 (as shown in FIG. 7) or partially reside in the interior cavity 704 (not shown). In the second case, the blade(s) at least partially extend out and away from the engagement member to which it is(are) coupled, and towards the other engagement member when the scissor forceps is in its closed position.

The blade(s) may be stationary or movable. In the later case, movement of the blade(s) in two opposing directions 710, 712 can be facilitated and/or controlled via an external device (not shown) (for example, a processor or computing device) communicatively and/or electrically coupled to the blade(s). The blade(s) may be disposed on a track 708. Tracks are well known and will not be described here. Power or other signal(s) can be provided to controller(s), motor(s) and/or gear(s) of the track via wire(s) 714, 716, 718.

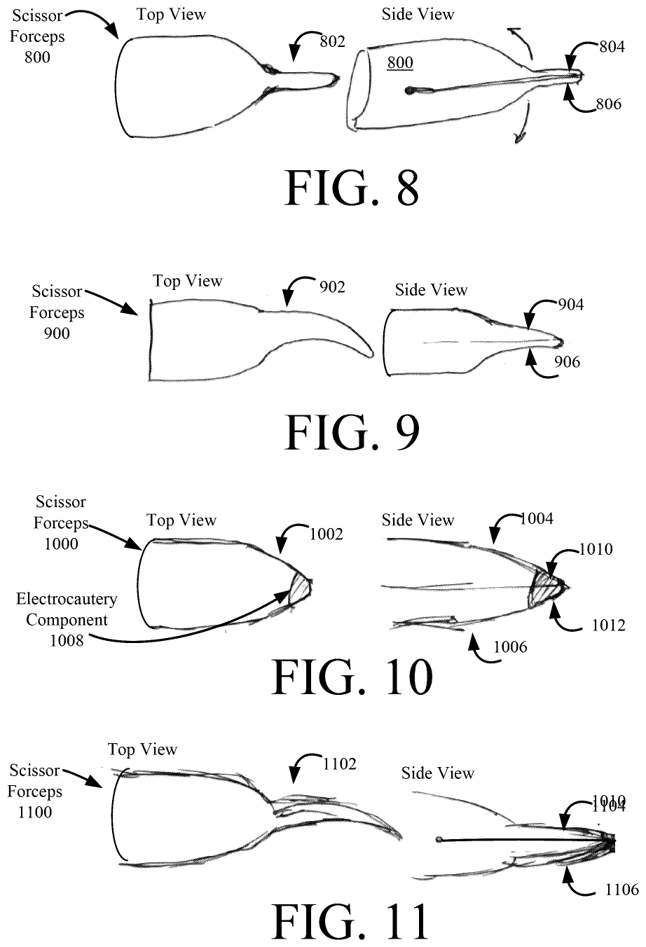

The overall design of the scissor forceps is not limited to that shown in FIGS. 1-7. The scissor forceps can have other shapes and/or sizes as shown in FIGS. 8-11. For example, as shown in FIG. 8, the engagement members 804, 806 of the scissor forceps 800 can be shaped and/or sized to provide a pointed or blunt dissector 802 for spread dissection of bronchial airwall enterotomy(-mies) and/or other purposes.

As shown in FIG. 9, the engagement members 904, 906 of the scissor forceps 900 can be shaped and/or sized to provide a curved Maryland dissector 902. The Maryland dissector 902 may have a fine tip for grabbing a small bit of tissue but a relatively long length allowing the engagement members 904, 906 to spread wide when used for blunt dissection.

As shown in FIG. 10, the engagement members 1004, 1006 of the scissor forceps 1000 can be shaped and/or sized to provide a dolphin dissector 1002. An electrocautery component 1008 may be provided on the tip of the dolphin dissector 1002. The electrocautery component 1008 can include one or more electrode(s) 1010, 1012. Reference numbers 1010, 1012 are pointing to a single electrode or two opposite polarity electrodes. The electrocautery component 1008 can be used to produce heat from electric current to destroy tissue (for example, a tumor) and/or control bleeding.

As shown in FIG. 11, the engagement members 1104, 1106 of the scissor forceps 1100 can be shaped and/or sized to provide a Metzenbaum style scissor 1102 for cutting. The tip of the scissor forceps 1100 may be slightly blunt.

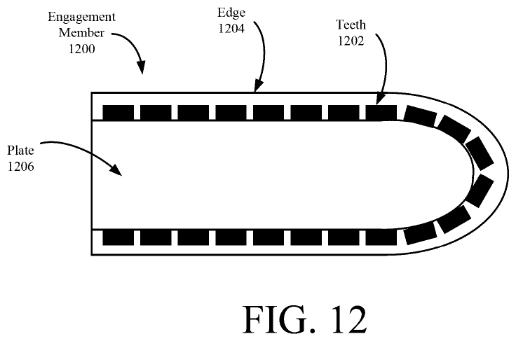

Referring now to FIG. 12, there is provided yet another architecture for an engagement member of the scissor forceps. The engagement member 1200 comprises teeth 1202 which extend along the periphery of the engagement member. The teeth 1200 may be offset from the edge 1204 of the engagement member 1200 by a given amount. A plate 1206 is provided within the engagement member 1200. The plate 1206 can be used to clamp and seal tissue within the scissor forceps. Additionally or alternatively, the plate 1206 can provide a means to pass current through the tissue clamped between itself and the other engagement members of the scissor forceps.

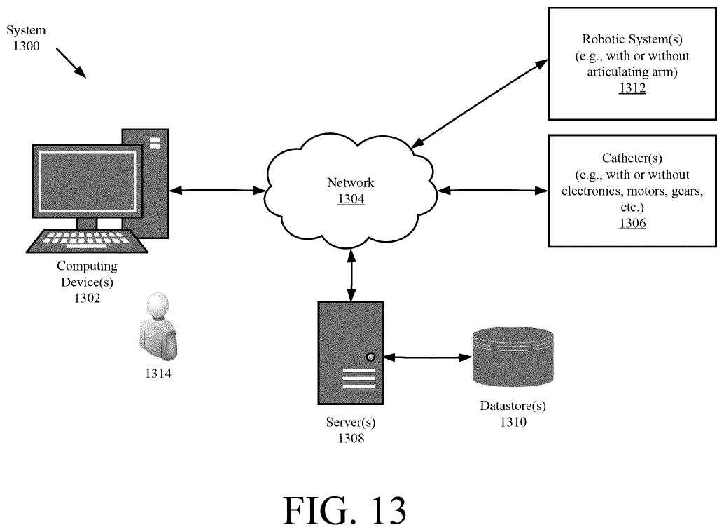

The above-described catheter can be manually operated, automatically operated and/or teleoperated. An illustrative system 1300 for automatically operating a catheter is provided in FIG. 13. System 1300 comprises computing device(s) 1302, server(s) 1308, datastore(s) 1310, robotic system(s) 1312 and catheter(s) 1306. Components 1302, 1306, 1308, 1312 are communicatively coupled to each other via network 1304 (for example, the Internet or an Intranet). An operator 1314 can control the robotic system 1312 and/or catheter(s) 1306 using a software application executing on computing device 1302.

The catheter(s) 1306 can comprise electronics, motors and/or gears. The electronics can include, but are not limited to, a processor, a computing device, a wireless communication device, and/or a power source. The wireless communication device allows command signal(s) to be wirelessly sent from the computing device(s) 1302 and/or robotic system(s) to the catheter(s) 1306 and/or component(s) (for example, sensor(s)) of the catheter(s) 1306.

The robotic system 1312 can comprise an articulating arm with a grasping mechanism on the distal end thereof. The grasping mechanism allows the robotic system to grasp and move the catheter in accordance with instructions from the operator 1314 via user software interactions with computing device 1302. The robotic system can alternatively or additionally comprise other operative components for handling, moving and/or otherwise operating the catheter and/or components thereof. These components can include, but are not limited to, sensors configured to generate sensor data. The sensor data may be stored in the datastore(s) 1310 for analysis.

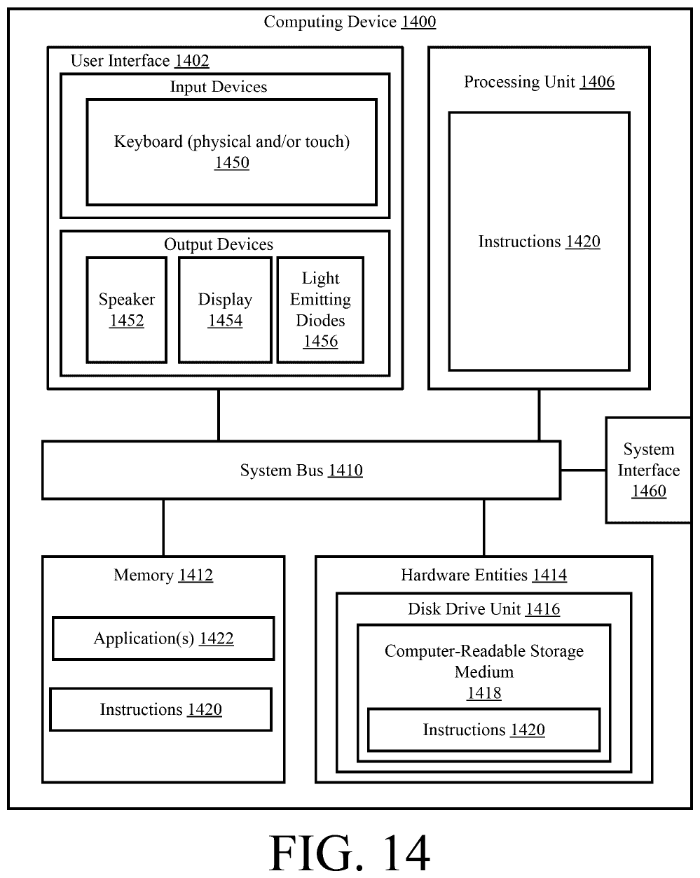

FIG. 14 provides a schematic illustration of a computing device 1400. Computing device 1302, server 1308 and/or robotic system 1312 of FIG. 13 can be the same as or similar to computing device 1400. As such, the discussion of computing device 1400 is sufficient for understanding components 1302, 1308, 1312 of FIG. 13.

The computing device 1400 may include more or less components than those shown in FIG. 14. However, the components shown are sufficient to disclose an illustrative embodiment implementing the present solution. The hardware architecture of FIG. 14 represents one embodiment of a computing device configured to facilitate the control of catheter(s) (for example, catheter 100 of FIGS. 1-3, 500 of FIG. 5, 600 of FIGS. 6 and/or 700 of FIG. 7), sensor(s) (for example, sensor(s) 504 of FIGS. 5 and/or 602 of FIG. 6), electrode(s) (for example, electrode(s) 602 of FIGS. 6 and/or 1010, 1012 of FIG. 10), fluid delivery/removal device(s) (for example, fluid delivery/removal device 504 of FIG. 5), and/or blade(s) (for example, blade 702 of FIG. 7).

Some or all the components of the computing device 1400 can be implemented as hardware, software and/or a combination of hardware and software. The hardware includes, but is not limited to, one or more electronic circuits. The electronic circuits can include, but are not limited to, passive components (e.g., resistors and capacitors) and/or active components (e.g., amplifiers and/or microprocessors). The passive and/or active components can be adapted to, arranged to and/or programmed to perform one or more of the methodologies, procedures, or functions described herein.

As shown in FIG. 14, the computing device 1400 comprises a user interface 1402, a Processing Unit (PU) 1406, a system bus 1410, a memory 1412 connected to and accessible by other portions of computing device 1400 through system bus 1410, and hardware entities 1414 connected to system bus 1410. The PU 1406 can include, but is not limited to, a Central Processing Unit (CPU) and/or a Graphical Processing Unit (GPU). The GPU may include graphical processing as well as algorithm processing for deep-learning or machine-learning actions. The user interface can include input devices (for example, a keypad 1450, mouse 1434 and microphone 1436) and output devices (e.g., speaker 1452, a display 1454, a vibration device 1458 and/or light emitting diodes 1456), which facilitate user-software interactions for controlling operations of the computing device 1400.

At least some of the hardware entities 1414 perform actions involving access to and use of memory 312, which can be a Random Access Memory (RAM), a disk driver and/or a Compact Disc Read Only Memory (CD-ROM).

Hardware entities 1414 can include a disk drive unit 1416 comprising a computer-readable storage medium 1418 on which is stored one or more sets of instructions 1420 (for example, software code) configured to implement one or more of the methodologies, procedures, or functions described herein. The instructions 1420 can also reside, completely or at least partially, within the memory 1412 and/or within the CPU 1406 during execution thereof by the computing device 1400. The memory 1412 and the CPU 1406 also can constitute machine-readable media. The term “machine-readable media”, as used here, refers to a single medium or multiple media (e.g., a centralized or distributed database, and/or associated caches and servers) that store the one or more sets of instructions 1420. The term “machine-readable media”, as used here, also refers to any medium that is capable of storing, encoding or carrying a set of instructions 1420 for execution by the computing device 1400 and that cause the computing device 1400 to perform any one or more of the methodologies of the present disclosure.

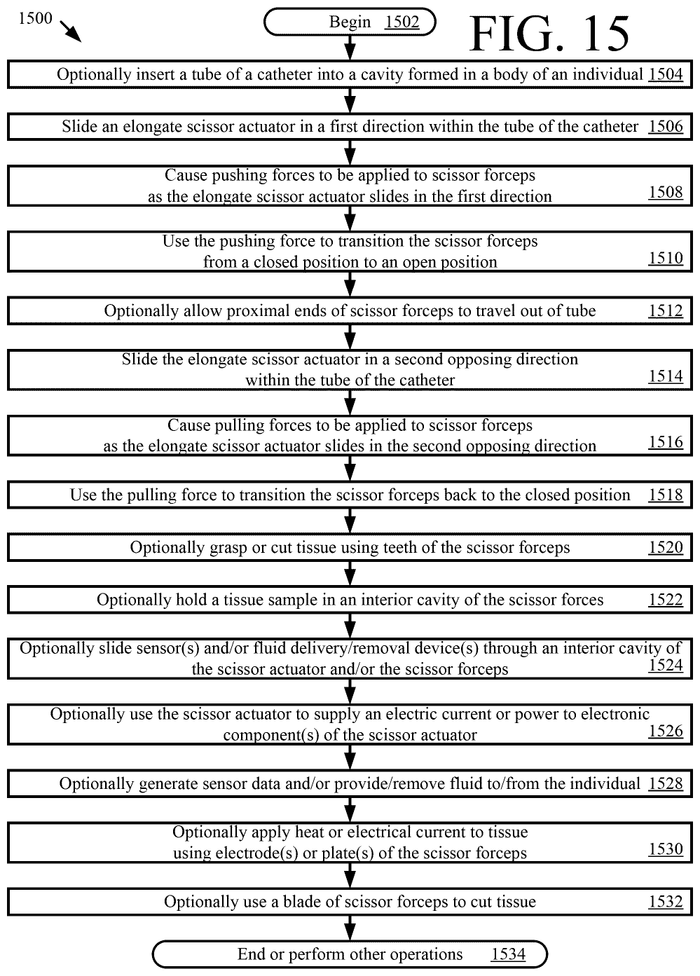

Referring now to FIG. 15, there is provided a flow diagram of an illustrative method 1500 for operating a catheter. Method 1500 begins with 1502 and continues with 1504 where a tube (for example, tube 102 of FIGS. 1-3) is inserted into a cavity formed in a body of an individual. Next, an elongate scissor actuator (for example, scissor actuator 200 of FIG. 2) is slid in a first direction (for example, direction 210 of FIG. 2) within the tube of the catheter, as shown by 1506. As the elongate scissor actuator slides in the first direction, pushing forces are applied to scissor forceps in 1508. The pushing forces are used in 1510 to transition the scissor forceps from a closed position (for example, the position shown in FIGS. 1-2) to an open position (for example, the position shown in FIG. 3). The scissor forceps comprise a pair of pivotally coupled engagement members (for example, engagement members 110, 112 of FIGS. 1-3) at least partially disposed within the tube at a distal end of the catheter. Each engagement member comprises a free distal end (for example, distal end 108 of FIG. 1) and a proximal end (for example, proximal end 214 of FIG. 2) coupled to the elongate scissor actuator. The proximal ends (for example, proximal ends 214 of FIG. 2) are optionally allowed to travel out from the tube (for example, as shown in FIG. 4).

In 1514, the scissor actuator is slid in a second opposing direction (for example, direction 212 of FIG. 2) within the tube of the catheter. Pulling forces are applied in 1516 to the scissor forceps as the scissor actuator is slide in the second opposing direction. The pulling forces are used in 1518 to transition the scissor forceps back to the closed position.

Method 1500 can then continue with one or more optional steps 1520-1532. These steps involve: grasping or cutting tissue using teeth (for example, teeth 114 of FIG. 1) of the scissor forceps; holding a tissue sample in an interior cavity (for example, interior cavity 122 of FIG. 1) of the scissor forceps; sliding sensor(s) and/or fluid delivery/removal device(s) (for example, device 504 of FIG. 5) through an interior cavity of the scissor actuator and scissor forceps; using the scissor actuator to supply an electric current or power to the electronic component(s) of the scissor actuator; generating sensor data by the sensor(s) of the scissor forceps; applying heat or electrical current to tissue using electrode(s) (for example, electrode(s) 602 of FIGS. 6 and/or 1010, 1012 of FIG. 10) coupled to the engagement member(s) of the scissor forceps; and/or using a blade (for example, blade 702 of FIG. 7) provided in an internal cavity of the engagement member(s) to cut tissue. Subsequently, 1534 is performed where method 1500 ends or other operations are performed (for example, return to 1502).

The present solution is not limited to the particular mechanical architecture described above. In other scenarios, the opening and closing of the engagement members can be facilitated using resilient members (for example, springs) and trigger mechanisms for actuating the resilient members (for example, a depressible button coupled to a linkage).

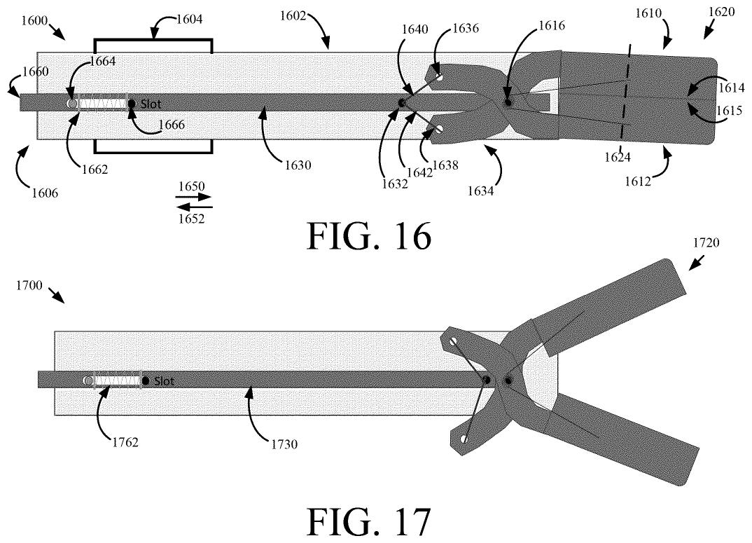

FIG. 16 provides illustrations of a catheter 1600. Catheter 1600 comprises a tube 1602 for being at least partially inserted through a narrow opening into a body cavity during a medical procedure. For example, the catheter 1600 may be used to remove fluid from a patient's bladder. The present solution is not limited to the particulars of this example.

The tube 1602 can be flexible or semi-rigid. The tube 1602 may be disposable or non-disposable. The tube 1602 may be formed of, for example, a rubber, a plastic, a polymer, an impregnated polymer, a cloth, a braided material and/or a metal. The metal can include, but is not limited to, stainless steel, titanium and/or other alloy which is resistant to corrosion.

A handle 1604 may optionally be provided with the catheter 1600. The handle 1604 resides at a proximal end 1606 of the tube 1602. The tube 1602 and handle 1604 may be integrally formed as one part (not shown), or alternatively may be coupled to each other via a coupling means as shown in FIGS. 16-17. The coupling means can include, but is not limited to, weld(s), adhesive(s), chemical bond(s) and/or mechanical coupler(s). In some scenarios, the handle is removably coupled to the tube 1602 such that it can be reused even when the tube is disposable. Accordingly, the mechanical coupler may comprise clamp(s), latch(es), and/or screw(s). The handle 1604 can be formed of any material suitable for a given application. The material can include, but is not limited to, rubber, plastic, polymer, and/or metal (for example, stainless steel, titanium and/or other alloy which is resistant to corrosion). The handle 1604 has a size and shape that allows an individual to easily grip and manipulate the catheter 1600 without discomfort.

Scissor forceps 120 are provided at a distal end 108 of the catheter 100. The scissor forceps 1620 may be formed of a rigid material such as plastic and metal. The metal can include, but is not limited to, stainless steel, titanium and/or other alloy which is resistant to corrosion. The scissor forceps 1620 are generally configured to grip and/or cut tissue of an individual. In this regard, the scissor forceps 1620 comprise engagement members 1610, 1612 which are pivotally coupled to each other via pivot element 1616 so that they are transitionable between a closed positions shown in FIG. 16 and an open position shown in FIG. 17. Center axis 1624 of the engagement members 1610, 1612 are aligned with each other when the scissor forceps 1620 are in the closed position as shown in FIG. 16.

In the closed position, surfaces 1614, 1616 of the engagement members 1610, 1612 abut and are in contact with each other. The present solution is not limited in this regard. For example, in other scenarios, the surfaces 1614, 1616 may be adjacent to one another but not necessarily contact each other. The surfaces 1614, 1616 may have teeth formed thereon or coupled thereto. Spacers may be provided to ensure that the opposing sets of teeth are spaced a certain distance from each other when the engagement members 1610, 1612 are in the closed position. The teeth may be dull or sharp depending on a given application for the catheter 1600. Each engagement member 1610, 1612 may be hollow such that an interior cavity is provided for holding and removing samples (for example, tissue samples) from the individual.

The scissor forceps 1620 are actuated by a scissor actuator 1630. Scissor actuator 1630 can include, but is not limited to, a post, a bar, a wire, a cable, a hollow tube and/or pin/slot configuration (for example, a pin at 1632 inserted into a slot on 1630). The scissor actuator 1630 is movable in two opposing directions 1650 and 1652, and pivotally coupled to proximal ends 1634 of the engagement members 1610, 1612 via pivot element 1632, 1636, 1638 and couplers 1640, 1642. Movement of the scissor actuator 1630 in direction 1650 is achieved via application of a pushing force on a free end 1660 of the scissor actuator 1630. Movement of the scissor actuator 1630 in direction 1650 causes the scissor forceps 1620 to open.

When the pushing force is no longer being applied to the free end 1660, the scissor actuator 1630 is automatically caused to travel in direction 1652 via a resilient member 1662. The resilient member 1662 may include, but is not limited to, a spring. The spring is normally in an uncompressed state and is compressed between pins 1664, 1666 when the scissor actuator 1630 is pushed in direction 1650. The spring transitions from its compressed state to its uncompressed state when the scissor actuator 1630 is released. This state transition of the spring causes the scissor actuator 1630 to travel in direction 1652, whereby the scissor forceps 1620 are caused to close.

Couplers 1640, 1642 can include, but are not limited to, wires, posts and/or plates. The couplers 1640, 1642 apply a pushing force respectively to the proximal ends 1634 of the engagement members 1610, 1612 so as to cause them to move away from each other when the scissor actuator 1630 slides in direction 1650. In contrast, the couplers 1640, 1642 apply a pulling force respectively to the proximal ends 1634 of the engagement members 1610, 1612 so as to cause them to move towards each other when the scissor actuator 1630 slides in direction 1652. The pushing and pulling forces facilitate transitioning of the engagement members 1610, 1612 between their open and closed positions.

The scissor forceps 1620 are coupled to the tube 1602 via any suitable means selected in accordance with a given application of the catheter 1600. In some scenarios, the scissor forceps 1620 are securely coupled to the tube 1602 by pin(s) (which may or may not comprise pivot element 1616). In other scenarios, the scissor forceps 1620 are removably coupled to the tube 1602 such that they can be reused even when the tube 1602 is disposable. This coupling of the scissor forceps 1620 to the tube 1602 can be achieved via pin(s) (which may or may not comprise pivot element 1616), a cap and/or stand-off structures.

The present solution is not limited to the particulars of FIG. 16. Another architecture for a catheter 1700 is provided in FIG. 17. Catheter 1700 is similar to catheter 1600 but is configured to operate in an opposite manner, i.e., the scissor forceps 1720 of catheter 1700 are normally in an open position (i.e., when the resilient member 1762 is in its uncompressed state) and transitions to its closed position via actuation of the scissor actuator 1730 (i.e., when the resilient member 1762 is compressed).

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A method for operating a catheter, comprising:
sliding a scissor actuator in a first direction within a tube of the catheter;
causing pushing forces to be applied to scissor forceps as the scissor actuator slides in the first direction;
using the pushing forces to transition the scissor forceps from a first position to a second position, the scissor forceps comprising a pair of pivotally coupled engagement members at least partially disposed within the tube at a distal end of the catheter, each engagement member comprising a free distal end and a proximal end coupled to the scissor actuator;
sliding the scissor actuator in a second opposing direction within the tube of the catheter;
causing pulling forces to be applied to the scissor forceps as the scissor actuator slides in a second opposing direction; and
using the pulling forces to transition scissor forceps back to the first position.

2. The method according to claim 1, further comprising allowing the proximal ends of the engagement members to travel outside of the tube when the scissor forceps is being transitioned to the second position.

3. The method according to claim 1, further comprising grasping or cutting tissue using teeth of the engagement members.

4. The method according to claim 3, further comprising holding a tissue sample in an interior cavity of at least one said engagement member.

5. The method according to claim 1, further comprising sliding at least one of a sensor, a fluid delivery device and a fluid removal device through an interior cavity of at least one of the scissor actuator and the scissor forceps.

6. The method according to claim 1, further comprising generating sensor data by a sensor at least partially disposed in at least one of the engagement members of the scissor forceps.

7. The method according to claim 1, further comprising applying heat or electrical current to tissue using one or more electrodes coupled to at least one of the engagement members of the scissor forceps.

8. The method according to claim 1, further comprising using a blade provided in an internal cavity of at least one of the engagement members to cut tissue.

9. The method according to claim 1, further comprising using the scissor actuator additionally to supply an electric current or power to at least one electronic component of the scissor actuator.

10. The method according to claim 1, further comprising using plates respectively disposed in the engagement members to deliver electrical current to tissue.

11. A catheter, comprising:
a tube;
a scissor actuator slidingly disposed in the tube; and
a scissor forceps comprising a pair of pivotally coupled engagement members at least partially disposed within the tube at a distal end of the catheter, each engagement member comprising a free distal end and a proximal end coupled to the scissor actuator;
wherein the scissor actuator is configured to cause pushing forces to be applied to the scissor forceps when the scissor actuator slides within the tube in a first direction, and pulling forces to be applied to the scissor forceps when the scissor actuator slides within the tube in a second opposing direction;
wherein the pushing forces cause the scissor forceps to transition from a first position to a second position, and the pulling forces cause the scissor forceps to transition back to the first position.

12. The catheter according to claim 11, wherein openings are formed in the tube through which the proximal ends of the engagement members travel out of the tube when the scissor forceps is being transitioned to the second position.

13. The catheter according to claim 11, wherein the engagement members comprise teeth for grasping or cutting tissue.

14. The catheter according to claim 13, wherein at least one of the engagement members comprises an interior cavity in which a tissue sample may be held.

15. The catheter according to claim 11, further comprising at least one of a sensor, a fluid delivery device and a fluid removal device that is slidingly disposed in an interior cavity of at least one of the scissor actuator and the scissor forceps.

16. The catheter according to claim 11, further comprising at least one sensor at least partially disposed in at least one of the engagement members of the scissor forceps.

17. The catheter according to claim 11, further comprising one or electrodes that are coupled to at least one of the engagement members of the scissor forceps and configured to apply heat or electrical current to tissue.

18. The catheter according to claim 11, further comprising a blade provided in an internal cavity of at least one of the engagement members.

19. The catheter according to claim 11, wherein the scissor actuator has a dual purpose of (i) actuating the scissor forceps and (ii) supplying an electric current or power to at least one electronic component of the scissor actuator.

20. The catheter according to claim 11, further comprising plates that are respectively disposed in the engagement members and configured to deliver electrical current to tissue.
